(19) United States
(12) Patent Application Publication
(10) Pub. No.: US 2023/0286972 A1
(43) Pub. Date: Sep. 14, 2023

(54) CRYSTALINE FORMS OF AN O-GLYCOPROTEIN-2-ACETAMIDO-2-DEOXY-3-D-GLUCOPYRANOSIDASE INHIBITOR

(71) Applicant: BIOGEN MA INC., Cambridge, MA (US)

(72) Inventors: Asmerom Weldeab, Melrose, MA (US); Tae Kim Correia, Raynham, MA (US); Aireal Diane Jenkins, Melrose, MA (US); Yiqing Lin, Lexington, MA (US); Chaomin Li, Boston, MA (US)

(21) Appl. No.: 18/019,270

(22) PCT Filed: Aug. 3, 2021
(86) PCT No.: PCT/US2021/044341

§ 371 Date: Feb. 2, 2023

Related U.S. Application Data
(60) Provisional application No. 63/060,281, filed on Aug. 3, 2020.

(51) Int. Cl. C07D 417/14

(52) U.S. Cl. CPC C07D 417/14; C07B 2200/13

(57) ABSTRACT

Described herein are solid forms of N-(4-fluoro-5-(((2S,4R)-4-46-methoxypyrimidin-4-yl)oxy)-2-methylpyrrolidin-1-yl)methyl)thiazol-2-yl)acetamide, compound (I): (I) and the process of making said solid forms of compound (I). The present invention further relates to a pharmaceutical composition comprising crystalline Form A and Form B of compound (I), and methods of using said form and pharmaceutical composition in the treatment and prevention of Alzheimer's disease and related neurological disorders.

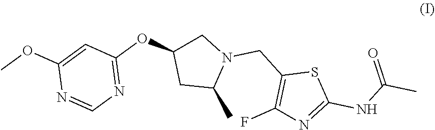 [SMILES: COc1cc(O[C@@H]2C[C@H](C)N(Cc3sc(NC(C)=O)nc3F)C2)ncn1]

### RELATED APPLICATION

This application claims the benefit of the filing date, under 35 U.S.C. § 119(e), of U.S. Provisional Application No. 63/060,281, filed on Aug. 3, 2020, the entire contents of which are incorporated herein by reference.

### FIELD OF INVENTION

The present invention generally relates to solid forms of N-(4-fluoro-5-(((2S,4R)-4-((6-methoxypyrimidin-4-yl)oxy)-2-methylpyrrolidin-1-yl)methyl)thiazol-2-yl)acetamide. The present invention further discloses the process for preparing said solid foul's, pharmaceutical compositions comprising said solid-forms, and methods of using said solid forms and pharmaceutical compositions thereof in the treatment or prevention of Alzheimer's disease or related neurodegenerative diseases.

### BACKGROUND

Alzheimer's disease (AD) is one of the most prevalent neurological disorders worldwide and the most common and debilitating age-related condition, causing progressive amnesia, dementia, and ultimately global cognitive failure and death. Currently, the only pharmacological therapies available are symptomatic drugs such as cholinesterase inhibitors or other drugs used to control the secondary behavioral symptoms of AD. Investigational treatments targeting the AD pathogenic cascade include those intended to inhibit the development of neurofibrillary tangles (NFTs).

A wide range of cellular proteins, both nuclear and cytoplasmic, are post-translationally modified by the addition of the monosaccharide 2-acetamido-2-deoxy-β-D-glucopyranoside ((3-N-acetyl glucosamine) which is attached via an O-glycosidic linkage. This monosaccharide is generally referred to as O-linked N-acetylglucosamine or O-GlcNAc. The enzyme responsible for post-translationally linking β-N-acetylglucosamine (GlcNAc) to specific serine and threonine residues of numerous nucleocytoplasmic proteins is O-GlcNAc transferase (OGTase). A second enzyme, known as O-glycoprotein-2-acetamido-2-deoxy-3-D-glucopyranosidase or O-GlcNAcase or OGA, removes this post-translational modification to liberate proteins, making the O-GlcNAc-modification a dynamic cycle occurring several times during the lifetime of a protein.

O-GlcNAc-modified proteins regulate a wide range of vital cellular functions including, e.g., transcription, proteasomal degradation and cellular signaling. O-GlcNAc is also found on many structural proteins, including the cytoskeletal protein “tau” which is responsible for stabilizing a key cellular network of microtubules that is essential for distributing proteins and nutrients within neurons. Importantly, tau has been clearly implicated in the etiology of several diseases including tauopathies, Alzheimer's disease, Parkinson's disease, dementia and cancer.

It is well established that Alzheimer's disease and a number of related tauopathies including Progressive Supranuclear Palsy (PSP) and amyotrophic lateral sclerosis (ALS) are characterized, in part, by the development of neurofibrillary tangles (NFTs). These NFTs are aggregates of paired helical filaments (PHFs) and are composed of an abnormal form of tau. In AD patients, tau becomes hyperphosphorylated, thereby disrupting its normal function, forming PHFs and ultimately aggregating to form NFTs.

Six isoforms of tau are found in the human brain. In AD patients, all six isoforms of tau are found in NFTs, and all are markedly hyperphosphorylated. Tau in healthy brain tissue bears only 2 or 3 phosphate groups, whereas those found in the brains of AD patients bear, on average, 8 phosphate groups.

It has recently emerged that increases in phosphorylation levels result in decreased O-GlcNAc levels and conversely, increased O-GlcNAc levels correlate with decreased phosphorylation levels. It has been shown that decreased glucose availability in brain leads to tau hyperphosphorylation. The gradual impairment of glucose transport and metabolism leads to decreased O-GlcNAc and hyperphosphorylation of tau (and other proteins). Accordingly, the inhibition of O-GlcNAcase, which prevents hyperphosphorylation of tau by preventing removal of O-GlcNac from tau, should compensate for the age-related impairment of glucose metabolism within the brains of health individuals as well as patients suffering from Alzheimer's disease or related neurodegenerative diseases.

However, a major challenge in developing inhibitors for blocking the function of mammalian glycosidases, including O-GlcNAcase, is the large number of functionally related enzymes present in tissues of higher eukaryotes. Accordingly, the use of non-selective inhibitors in studying the cellular and organismal physiological role of one particular enzyme is complicated because complex phenotypes arise from the concomitant inhibition of such functionally related enzymes. In the case of β-N-acetylglucosaminidases, existing compounds that act to block O-GlcNAcase (OGA) function are non-specific and act potently to inhibit the lysosomal β-hexosaminidases.

Orally active OGA inhibitors have been previously described in PCT/US2019/051661. However, after a specific compound is identified as a promising candidate for use in a pharmaceutical composition, it is still necessary to fine-tune its properties with respect to a number of critical parameters, such as stability in solid state and/or liquid formulations, hygroscopicity, crystallinity, toxicological considerations, melting point, or solubility in water and aqueous media.

In view of foregoing technical challenge and given the potential for regulation of O-GlcNAcase for treatment of AD, tauopathies and other neurological diseases, there remains a need for discovery of potent solid forms of O-GlcNAcase inhibitors.

### SUMMARY

The present disclosure provides different forms of the Compound (I)

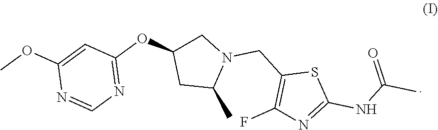 [SMILES: COc1cc(O[C@@H]2C[C@H](C)N(Cc3sc(NC(C)=O)nc3F)C2)ncn1]

Embodiments of these crystalline forms include those characterized forms A and B. The names used herein to characterize a specific form, e.g. Form A and Form B should not be considered limiting with respect to any other substance possessing similar or identical physical and chemical characteristics, but rather it should be understood that these designations are mere identifiers that should be interpreted according to the characterization information also presented herein.

In another aspect, provided herein is a pharmaceutical composition comprising the crystalline Form A of Compound (I) and at least one pharmaceutically acceptable carrier or diluent.

In another aspect, provided herein is the crystalline Form A of the Compound (I) for use as a medicament.

In a further aspect, provided herein is the crystalline Form A of the Compound (I) for use in the treatment or prevention of Alzheimer's disease or a related neurological disease.

In a further aspect, provided herein is a process of manufacturing the crystalline Form A of the Compound (f).

In another aspect, provided herein is a pharmaceutical composition comprising the crystalline Form B of the Compound (I) and at least one pharmaceutically acceptable carrier or diluent.

In another aspect, provided herein is the crystalline Form B of the Compound (I) for use as a medicament.

In a further aspect, provided herein is the crystalline Form B of the Compound (I) for use in the treatment or prevention of Alzheimer's disease or a related neurological disease.

In a further aspect, provided herein is a process of manufacturing the crystalline Form A of the Compound (I).

### BRIEF DESCRIPTION OF THE DRAWINGS

FIG. 1: shows the X-ray powder diffraction pattern for freeform Type A of Compound (I).

FIG. 1B: shows the TGA/DSC curves of freeform Type A of Compound (I).

FIG. 2: shows the X-ray powder diffraction pattern for Freeform type B of Compound (I).

FIG. 2B: shows the TGA/DSC curves for Freeform Type B of Compound (I).

FIG. 3: shows the X-ray powder diffraction pattern for Amorphous Freeform of Compound (I).

FIG. 4: shows the X-ray powder diffraction pattern for HCl salt Form A of Compound (I).

FIG. 4B: shows the TGA/DSC curves for HCl salt Form A of Compound (I).

FIG. 5: shows the X-ray powder diffraction pattern for phosphate salt Fort A of Compound (I).

FIG. 5B: shows the TGA/DSC curves for phosphate salt Form A of the compound (I).

FIG. 6: shows the X-ray powder diffraction pattern for Tartrate salt Form B of Compound (I).

FIG. 6B: shows the TGA/DSC curves for Tartrate salt Form B of Compound (I).

FIG. 7: shows the X-ray powder diffraction pattern for Tartrate salt Form A of Compound (I).

FIG. 7B: shows the TGA/DSC curves for Tartrate salt Form A of Compound (I).

FIG. 8: shows the X-ray powder diffraction pattern for Tartrate salt Form C of Compound (I).

FIG. 8B: shows the TGA/DSC curves for Tartrate salt Form of the Compound (I).

FIG. 9: shows the X-ray powder diffraction pattern for Tartrate salt Form D of Compound (I).

FIG. 9B: shows the TGA/DSC curves for Tartrate salt Form D of the Compound (I).

FIG. 10: shows the X-ray powder diffraction pattern for HBr salt Form A of Compound (I).

FIG. 10B: shows the TGA/DSC curves for HBr salt Form A of the Compound (I).

FIG. 11: shows the X-ray powder diffraction pattern for Fumarate salt Form A of Compound (I).

FIG. 11B: shows the TGA/DS curves for Fumarate salt Form A of Compound (I).

FIG. 12: shows the X-ray powder diffraction pattern for Fumarate salt Form B of Compound (I).

FIG. 12B: shows the TGA/DSC curves for Fumarate salt Form B of Compound (I).

FIG. 13: shows the X-ray powder diffraction pattern for Fumarate salt Form C of Compound (I).

FIG. 13B: shows the TGA/DSC curves for Fumarate salt Form C of Compound (I).

FIG. 14: shows the X-ray powder diffraction pattern for Fumarate salt Form D of Compound (I).

FIG. 14B: shows the TGA/DSC curves for Fumarate salt Form D of Compound (I).

FIG. 15: shows the X-ray powder diffraction pattern for Fumarate salt Form E of Compound (I).

FIG. 15B shows the TGA/DSC curves for Fumarate salt Form F of Compound (I).

FIG. 16: shows the X-ray powder diffraction pattern for Fumarate salt Form F of Compound (I).

FIG. 16B: shows the TGA/DSC curves for Fumarate salt Form F of Compound (I).

FIG. 17: shows the X-ray powder diffraction pattern for Fumarate salt Form G of Compound (I).

FIG. 1713: shows the TGA/DSC curves for Fumarate salt Form G of Compound (I).

### DETAILED DESCRIPTION OF THE INVENTION

In one aspect, provided herein is a crystalline Form A of the Compound (I).

In another aspect, provided herein is a crystalline Form B of the Compound (I).

The present invention provides a polymorphic form of N-(4-fluoro-5-(((2S,4R)-4-((6-methoxypyrimidin-4-yl)oxy)-2-methylpyrrolidin-1-yl)methyl)thiazol-2-yl)acetamide, which is Form A. N-(4-fluoro-5-((2S,4R)-4-((6-methoxypyrimidin-4-yl)oxy)-2-methylpyrrolidin-1-yl)methyl)thiazol-2-yl)acetamide, also referred to as the “Compound of Formula 1” or “Compound (T)”, or “Compound 1”, was originally described in PCT/US2019/051661 Example 1-22. PCT/US2019/051661 is incorporated herewith by reference in its entirety, in particular the disclosure related to the synthesis of Example 1-22.

As described herein, the free base of Compound 1 can be a crystalline form that exists as one or more polymorph forms, including anhydrate forms. These polymorph forms (alternatively known in the art as polymorphic forms or crystal forms) differ with respect to their X-ray powder diffraction patterns, spectroscopic, physicochemical and pharmacokinetic properties, as well as their thermodynamic stability.

It is desirable to have access to different polymorphic forms of Compound 1 for several reasons. Distinct polymorph forms may exhibit different physical properties such as melting point, hygroscopicity, solubility, flow properties or thermodynamic stability, and therefore, distinct polymorph forms allow the choice of the most suitable form for a given use or aspect, for example, in distinct administration forms such as capsules, or in the manufacture of a drug form having optimum pharmacokinetic properties.

It has now been surprisingly found that under certain conditions new solid forms of N-(4-fluoro-5-(((2S,4R)-4-((6-methoxypyrimidin-4-yl)oxy)-2-methylpyrrolidin-1-yl)methyl)thiazol-2-yl)acetamide, can be provided which are described hereinafter as Form A, Form B, and amorphous form, and which have advantageous utilities and properties. In particular, Form A of the Compound of Formula 1 shows excellent stability properties when subject to stress conditions. A particular polymorph form of Compound 1, namely Form A, is more stable than all other solid forms of Compound 1 disclosed herein. This high degree of stability of Form A provides advantageous properties and benefits in terms of its suitability for use in a pharmaceutical composition, for example, in terms of its shelf-life and ease of manufacture.

The invention provides the crystalline Form A of N-(4-fluoro-5-(((2S,41Z)-4-((6-methoxypyrimidin-4-yl)oxy)-2-methylpyrrolidin-1-yl)methyl)thiazol-2-yl)acetamide, (Compound 1) in free form. The term “free form” refers to the compound per se without salt formation.

Also disclosed herein are anhydrate Free Form A, anhydrate tartrate salt Form B, anhydrate HCl salt Form A and anhydrate Phosphate salt Form A, anhydrate HBr salt Form A, anhydrate Fumarate salt Form A, B, C, D, E, F, G font in free form.

Also disclosed herein is anhydrate Form B

Also disclosed herein are anhydrate Tartrate Form A, C, D.

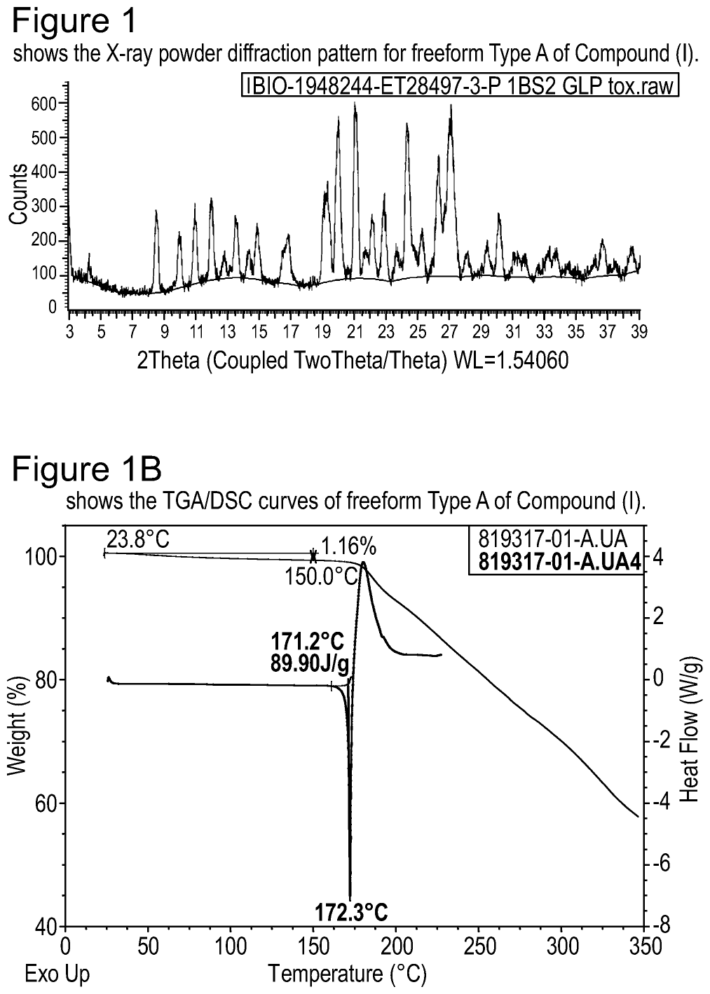

In one embodiment, the Compound of Formula 1 is crystalline Form A. Crystalline Form A can be defined by reference to one or more characteristic signals that result from analytical measurements including, but not limited to: X-ray powder diffraction pattern of FIG. 1, the differential scanning calorimetry (TGA/DSC) thermogram of FIG. 1B. Crystalline Form A (also referred to herein as polymorph Form A) can also be defined by reference to one or more of the following characteristic signals:

In one embodiment, the crystalline Form A has an X-ray powder diffraction pattern with at least one, two or three peaks having angle of refraction 2 theta (Θ) values selected from 4.3, 8.6 and 12.0° when measured using CuKa radiation, wherein said values are plus or minus 0.2° 2Θ.

In one embodiment, the crystalline Form A has an X-ray powder diffraction pattern with at least one, two or three peaks having angle of refraction 2 theta (Θ) values selected from 10, 11 and 19.9° when measured using CuKa radiation, wherein said values are plus or minus 0.2° 2Θ.

In one embodiment, the crystalline Form A has an X-ray powder diffraction pattern with at least one, two or three peaks having angle of refraction 2 theta (Θ) values selected from 13,5, 14,9, 21.1, 24.4 and 27.2° when measured using CuKa radiation, wherein said values are plus or minus 0.2° 2Θ.

In one embodiment, the crystalline Form A has an X-ray powder diffraction pattern with at least one, two, three, four or five peaks having angle of refraction 2 theta (Θ) values selected from 4.3, 8.6, 10, 11, 12, 13.5, 14.9, 19.9, 21.1, 24.4° when measured using CuKa radiation, wherein said values are plus or minus 0.2° 2Θ.

In one embodiment, crystalline Form A of the Compound of Formula 1 exhibits an X-ray powder diffraction pattern substantially the same as the X-ray powder diffraction pattern shown in FIG. 1 when measured using CuKa radiation.

In a further embodiment, crystalline Form A of the Compound of Formula 1 exhibits a differential scanning calorimetry (DSC) thermogram substantially the same as that shown in shown in FIG. 1B.

In a further embodiment, crystalline Form A of the Compound of Formula 1 exhibits a differential scanning calorimetry (DSC) thermogram with an onset of melting of about 171° C.

In one embodiment of the invention, there is provided crystalline Form A of N-(4-fluoro-5-(((2S,4R)-4-((6-methoxypyrimidin-4-yl)oxy)-2-methylpyrrolidin-1-yl)methyl)thiazol-2-yl)acetamide, in substantially pure form.

As used herein, “substantially pure,” when used in reference to crystalline forms and amorphous form of N-(4-fluoro-5-(((2S,4R)-44(6-methoxypyrimidin-4-yl)oxy)-2-methylpyrrolidin-1-yl)methyl)thiazol-2-yl)acetamide, means having a purity greater than 90 weight %, including greater than 90, 91, 92, 93, 94, 95, 96, 97, 98, and 99 weight and also including equal to about 100 weight % of N-(4-fluoro-5-(((2S,4R)-4-((6-methoxypyrimidin-4-yl)oxy)-2-methylpyrrolidin-1-yl)methyl)thiazol-2-yl)acetamide, based on the weight of the compound.

in another embodiment, the Compound of Formula 1 is freeform. Freeform can be defined by reference to one or more characteristic signals that result from analytical measurements including, but not limited to: X-ray powder diffraction pattern of FIG. 1. Freeform can also be defined by reference to one or more of the following characteristic signals: In one embodiment, Form A has an X-ray powder diffraction pattern with at least one, two or three peaks having angle of refraction 2 theta (Θ) values selected from 12, 19.9, 24.4° when measured using CuKa radiation, wherein said values are plus or minus 0.2° 2Θ.

In one embodiment, the freeform A has an X-ray powder diffraction pattern with at least one, two or three peaks having angle of refraction 2 theta (Θ) values selected from 4.3, 8.6, 19.9, 21.1, 24.4° when measured using CuKa radiation, wherein said values are plus or minus 0.2° 2Θ,

In one embodiment, the freeform A has an X-ray powder diffraction pattern with at least one, two, three, four or five peaks having angle of refraction 2 theta (Θ) values selected from 4.3, 8.6, 10, 11, 12, 13.5, 14.9, 19.9, 21.1, 244° when measured using CuKa radiation, wherein said values are plus or minus 0.2° 2Θ.

In one embodiment, freeform Form A of the Compound of Formula 1 exhibits an X-ray powder diffraction pattern substantially the same as the X-ray powder diffraction pattern shown in FIG. 1 when measured using CuKa radiation.

The term “substantially the same” with reference to X-ray diffraction peak positions means that typical peak position and intensity variability are taken into account. For example, one skilled in the art will appreciate that the peak positions (2Θ) will show some inter-apparatus variability, typically as much as 0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only. An expression referring to a crystalline Form A having “an X-ray powder diffraction pattern substantially the same as the X-ray powder diffraction pattern shown in FIG. 1” may be interchanged with an expression referring to a crystalline Form A having “an X-ray powder diffraction pattern characterized by the representative X-ray powder diffraction pattern shown in FIG. 1”.

One of ordinary skill in the art will also appreciate that an X-ray diffraction pattern may be obtained with a measurement error that is dependent upon the measurement conditions employed. In particular, it is generally known that intensities in an X-ray diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional X-ray diffraction pattern is typically about 5% or less, and such degree of measurement error should be taken into account as pertaining to the aforementioned diffraction angles. Consequently, it is to be understood that the crystal form of the instant invention is not limited to the crystal form that provides an X-ray diffraction pattern completely identical to the X-ray diffraction pattern depicted in the accompanying FIG. 1 disclosed herein. Any crystal forms that provide X-ray diffraction patterns substantially identical to that disclosed in the accompanying FIG. 1 fall within the scope of the present invention. The ability to ascertain substantial identities of X-ray diffraction patterns is within the purview of one of ordinary skill in the art.

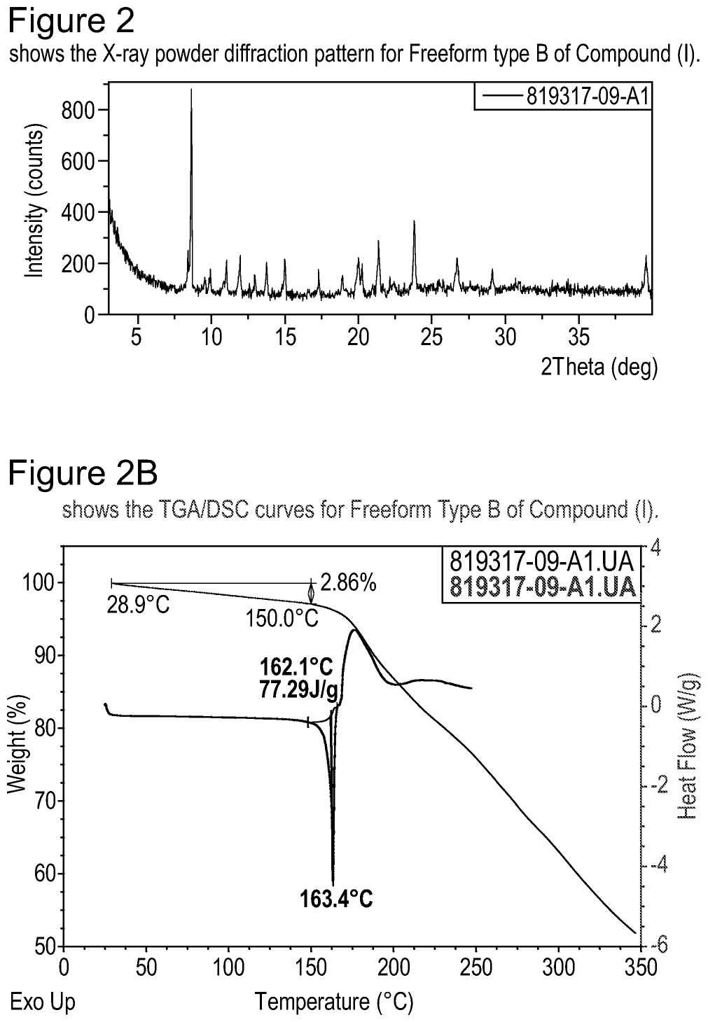

Crystalline Form B can be defined by reference to one or more characteristic signals that result from analytical measurements including, but not limited to: X-ray powder diffraction pattern of FIG. 2, the differential scanning calorimetry (DSC) thermogram of FIG. 2B. Crystalline Form 13 (also referred to herein as polymorph Form B) can also be defined by reference to one or more of the following characteristic signals: The crystalline Form B has an X-ray powder diffraction pattern with at least one, two or three peaks having angle of refraction 2 theta (Θ) values selected from 8.6, 11.1, 15.0° when measured using CuKa radiation, wherein said values are plus or minus 0.2° 2Θ.

The crystalline Form B has an X-ray powder diffraction pattern with at least one, two or three peaks having angle of refraction 2 theta (Θ) values selected from 8.6, 11.1, 12.0, 13.7, 15.0° when measured using CuKa radiation, wherein said values are plus or minus 0.2° 2Θ.

The crystalline Form B has an X-ray powder diffraction pattern with at least one, two, three, four or five peaks having angle of refraction 2 theta (Θ) values selected from 8.6, 9.5, 9.9, 11.1, 12.0, 13.7, 15.0, 21.5, 23.8° when measured using CuKa radiation, wherein said values are plus or minus 0.2° 2Θ.

The crystalline Form B of the Compound of Formula 1 exhibits an X-ray powder diffraction pattern substantially the same as the X-ray powder diffraction pattern shown in FIG. 2 when measured using CuKa radiation.

The crystalline Form B of the Compound of Formula 1 exhibits a differential scanning calorimetry (DSC) thermogram substantially the same as that shown in shown in FIG. 2B.

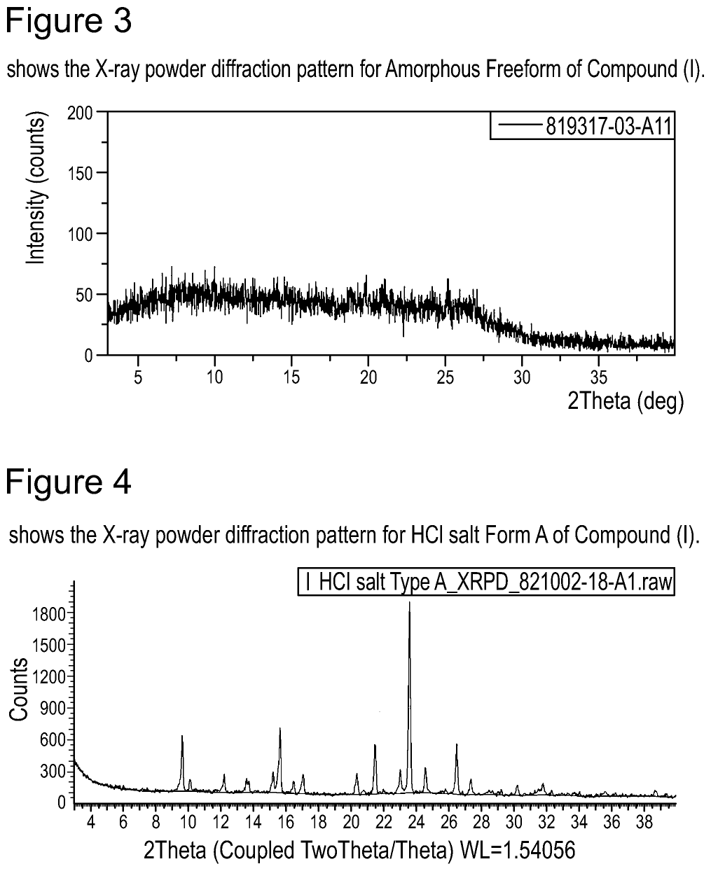

The amorphous form can be defined by analytical measurements including, but not limited to reference to an XRPD pattern substantially the same as the pattern shown in FIG. 3.

Seed crystals may be added to any crystallization mixture to promote crystallization. Seeding may be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971, 26, 369-377. In general, seeds of small size are needed to control effectively the growth of crystals in the batch. Seed of small size may be generated by sieving, milling, or micronizing of large crystals, or by micro-crystallization of solutions. Care should be taken that milling or micronizing of crystals does not result in any change in crystallinity form the desired crystal form (i.e., change to amorphous or to another polymorph).

### Method of Treatment

The present invention also provides a method for the treatment or prevention of diseases, conditions and/or disorders modulated by OGA inhibition, for example such as indicated herein, in a subject in need of such treatment or prevention, which method comprises administering to said subject a therapeutically effective amount of a crystalline Form of a Compound of Formula 1.

In one embodiment of the method, the OGA inhibition is inhibition of 0-GlcNAcase.

In another embodiment of the method, the disease or disorder is Alzheimer's disease or a related neurological disorder.

In one embodiment, the present invention provides the use of crystalline Form A of the Compound of Formula 1 for the manufacture of a medicament for the treatment or prevention of Alzheimer's disease or related neurodegenerative diseases.

In another aspect, provided herein is crystalline Form A of the Compound of Formula 1 for use as a medicament.

In a further aspect, provided herein is crystalline Form A of the Compound of Formula 1 for use in the treatment or prevention of Alzheimer's disease or related neurodegenerative diseases.

In another embodiment, the present invention provides the use of crystalline ICI Form A of the Compound of Formula 1 for the manufacture of a medicament for the treatment or prevention of Alzheimer's disease or related neurodegenerative diseases.

In another aspect, provided herein is crystalline HCl Form A of the Compound f Formula 1 for use as a medicament.

In a further aspect, provided herein is crystalline HCl Form A of the Compound of Formula 1 for use in the treatment or prevention of Alzheimer's disease or related neurodegenerative diseases.

In another embodiment, the present invention provides the use of crystalline phosphate Form A of the Compound of Formula 1 for the manufacture of a medicament for the treatment or prevention of Alzheimer's disease or related neurodegenerative diseases.

In another aspect, provided herein is crystalline Form phosphate A of the Compound of Formula 1 for use as a medicament.

In a further aspect, provided herein is crystalline phosphate Form A of the Compound of Formula 1 for use in the treatment or prevention of Alzheimer's disease or related neurodegenerative diseases.

In one embodiment, the present invention provides the use of crystalline Form B of the Compound of Formula 1 for the manufacture of a medicament for the treatment or prevention of Alzheimer's disease or related neurodegenerative diseases.

In another aspect, provided herein is crystalline Form 13 of the Compound of Formula 1 for use as a medicament.

In a further aspect, provided herein is crystalline Form 13 of the Compound of Formula 1 for use in the treatment or prevention of Alzheimer's disease or related neurodegenerative diseases.

In one embodiment, the present invention provides the use of crystalline Tartrate Form B of the Compound of Formula 1 for the manufacture of a medicament for the treatment or prevention of Alzheimer's disease or related neurodegenerative diseases.

In another aspect, provided herein is crystalline Tartrate Form B of the Compound of Formula 1 for use as a medicament.

In a further aspect, provided herein is crystalline Tartrate Form B of the Compound of Formula 1 for use in the treatment or prevention of Alzheimer's disease or related neurodegenerative diseases.

In one embodiment, the present invention provides the use of crystalline Tartrate Form A of the Compound of Formula 1 for the manufacture of a medicament for the treatment or prevention of Alzheimer's disease or related neurodegenerative diseases,

In another aspect, provided herein is crystalline Tartrate Form A of the Compound of Formula 1 for use as a medicament.

In a further aspect, provided herein is crystalline Tartrate Form A of the Compound of Formula 1 for use in the treatment or prevention of Alzheimer's disease or related neurodegenerative diseases.

In one embodiment, the present invention provides the use of crystalline Tartrate Form C of the Compound of Formula 1 for the manufacture of a medicament for the treatment or prevention of Alzheimer's disease or related neurodegenerative diseases.

In another aspect, provided herein is crystalline Tartrate Form C of the Compound of Formula 1 for use as a medicament.

In a further aspect, provided herein is crystalline Tartrate Form C of the Compound of Formula 1 for use in the treatment or prevention of Alzheimer's disease or related neurodegenerative diseases.

In one embodiment, the present invention provides the use of crystalline Tartrate Form D of the Compound of Formula 1 for the manufacture of a medicament for the treatment or prevention of Alzheimer's disease or related neurodegenerative diseases.

In another aspect, provided herein is crystalline Tartrate Form D of the Compound of Formula 1 for use as a medicament,

In a further aspect, provided herein is crystalline Tartrate Form D of the Compound of Formula 1 for use in the treatment or prevention of Alzheimer's disease or related neurodegenerative diseases.

In one embodiment, the present invention provides the use of crystalline HBr salt Form A of the Compound of Formula 1 for the manufacture of a medicament for the treatment or prevention of Alzheimer's disease or related neurodegenerative diseases.

In another aspect, provided herein is crystalline HBr salt Form A of the Compound of Formula 1 for use as a medicament.

In a further aspect, provided herein is crystalline HBr salt Form A of the Compound of Formula 1 for use in the treatment or prevention of Alzheimer's disease or related neurodegenerative diseases.

In one embodiment, the present invention provides the use of crystalline Fumarate salt Form A of the Compound of Formula 1 for the manufacture of a medicament for the treatment or prevention of Alzheimer's disease or related neurodegenerative diseases.

In another aspect, provided herein is crystalline Fumarate salt Form A of the Compound of Formula 1 for use as a medicament.

In a further aspect, provided herein is crystalline Fumarate salt Form A of the Compound of Formula 1 for use in the treatment or prevention of Alzheimer's disease or related neurodegenerative diseases.

In one embodiment, the present invention provides the use of crystalline Fumarate salt Form B of the Compound of Formula 1 for the manufacture of a medicament for the treatment or prevention of Alzheimer's disease or related neurodegenerative diseases.

In another aspect, provided herein is crystalline Fumarate salt Form B of the Compound of Formula 1 for use as a medicament.

In a further aspect, provided herein is crystalline Fumarate salt Form B of the Compound of Formula 1 for use in the treatment or prevention of Alzheimer's disease or related neurodegenerative diseases.

In one embodiment, the present invention provides the use of crystalline Fumarate salt Form C of the Compound of Formula 1 for the manufacture of a medicament for the treatment or prevention of Alzheimer's disease or related neurodegenerative diseases.

In another aspect, provided herein is crystalline Fumarate salt Form C of the Compound of Formula 1 for use as a medicament.

In a further aspect, provided herein is crystalline. Fumarate salt Form C of the Compound of Formula 1 for use in the treatment or prevention of Alzheimer's disease or related neurodegenerative diseases.

In one embodiment, the present invention provides the use of crystalline Fumarate salt Form D of the Compound of Formula 1 for the manufacture of a medicament for the treatment or prevention of Alzheimer's disease or related neurodegenerative diseases.

In another aspect, provided herein is crystalline Fumarate salt Form D of the Compound of Formula 1, for use as a medicament.

In a further aspect, provided herein is crystalline Fumarate salt Form D of the Compound of Formula 1 for use in the treatment or prevention of Alzheimer's disease or related neurodegenerative diseases.

In one embodiment, the present invention provides the use of crystalline Fumarate salt Form E of the Compound of Formula 1 for the manufacture of a medicament for the treatment or prevention of Alzheimer's disease or related neurodegenerative diseases.

In another aspect, provided herein is crystalline Fumarate salt Form E of the Compound of Formula 1 for use as a medicament.

In a further aspect, provided herein is crystalline Fumarate salt Form E of the Compound of Formula 1 for use in the treatment or prevention of Alzheimer's disease or related neurodegenerative diseases.

In one embodiment, the present invention provides the use of crystalline Fumarate salt Form F of the Compound of Formula 1 for the manufacture of a medicament for the treatment or prevention of Alzheimer's disease or related neurodegenerative diseases.

In another aspect, provided herein is crystalline Fumarate salt Form F of the Compound of Formula 1 for use as a medicament.

In a further aspect, provided herein is crystalline Fumarate salt Form F of the Compound of Formula 1 for use in the treatment or prevention of Alzheimer's disease or related neurodegenerative diseases.

In one embodiment, the present invention provides the use of crystalline Fumarate salt Form G of the Compound of Formula 1 for the manufacture of a medicament for the treatment or prevention of Alzheimer's disease or related neurodegenerative diseases.

In another aspect, provided herein is crystalline Fumarate salt Form G of the Compound of Formula 1 for use as a medicament.

In a further aspect, provided herein is crystalline. Fumarate salt Form G of the Compound of Formula 1 for use in the treatment or prevention of Alzheimer's disease or related neurodegenerative diseases.

Also provided is a method of treating a subject with a disease or condition selected from a neurodegenerative disease, a tauopathy, diabetes, cancer and stress, comprising administering to the subject an effective amount of the compound described herein, or a pharmaceutically acceptable salt thereof, or an effective amount of a pharmaceutical composition comprising at least one compound described herein, or a pharmaceutically acceptable salt thereof, and at least one pharmaceutically acceptable excipient.

Also provided is a method of inhibiting O-GlcNAcase in a subject in need thereof, comprising administering to the subject an effective amount of the compound described herein, or a pharmaceutically acceptable salt thereof, or an effective amount of a pharmaceutical composition comprising at least one compound described herein, or a pharmaceutically acceptable salt thereof, and at least one pharmaceutically acceptable excipient.

Also provided is a method of treating a disease or condition characterized by hyperphosphorylation of tau in the brain, comprising administering to the subject an effective amount of the compound described herein, or a pharmaceutically acceptable salt thereof, or an effective amount of a pharmaceutical composition comprising at least one compound described herein, or a pharmaceutically acceptable salt thereof, and at least one pharmaceutically acceptable excipient. In one embodiment, the disease or condition characterized by hyperphosphorylation of tau in the brain is Alzheimer's disease.

One aspect of the invention includes a method for treating a disease or a condition that is caused, mediated and/or propagated by O-GlcNAcase activity in a subject, the method comprising administering to the subject a therapeutically effective amount of compound (I), or a pharmaceutically acceptable salt thereof. Preferably, the disease or condition is a neurological disorder, diabetes, cancer or stress. More preferably, the disease or condition is a neurological disorder. In one embodiment, the neurological disorder is one or more tauopathies selected from Acute ischemic stroke (AIS), Alzheimer's disease, Dementia, Amyotrophic lateral sclerosis (ALS), Amyotrophic lateral sclerosis with cognitive impairment (ALSci), Argyrophilic grain dementia, Bluit disease, Corticobasal degeneration (CBP), Dementia pugilistica, Diffuse neurofibrillary tangles with calcification, Down's syndrome, epilepsy, Familial British dementia, Familial Danish dementia, Frontotemporal dementia with parkinsonism linked to chromosome 17 (FTDP-17), Gerstmann-Straussler-Scheinker disease, Guadeloupean parkinsonism, Hallevorden-Spatz disease (neurodegeneration with brain iron accumulation type 1), ischemic stroke, mild cognitive impairment (MCI), Multiple system atrophy, Myotonic dystrophy, Niemann-Pick disease (type C), Pallido-ponto-nigral degeneration, Parkinsonism-dementia complex of Guam, Pick's disease (PiD), Postencephalitic parkinsonism (PEP), Prion diseases (including Creutzfeldt-Jakob Disease (GJD), Variant Creutzfeldt-Jakob Disease (vCJD), Fatal Familial Insomnia, Kuru, Progressive supercortical gliosis, Progressive supranuclear palsy (PSP), Steele-Richardson-Olszewski syndrome, Subacute sclerosing panencephalitis, Tangle-only dementia, Huntington's disease, and Parkinson's disease. In another embodiment, the neurological disorder is one or more tauopathies selected from Acute ischemic stroke (AIS), Alzheimer's disease, Dementia, Amyotrophic lateral sclerosis (ALS), Amyotrophic lateral sclerosis with cognitive impairment (ALSci), Argyrophilic grain dementia, epilepsy, mild cognitive impairment (MCI), Huntington's disease, and Parkinson's disease. In yet another embodiment, the neurological disorder is Alzheimer's disease.

As used herein, the term “subject” and “patient” may be used interchangeably, and means a mammal in need of treatment, e.g., companion animals (e.g., dogs, cats and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or ‘treatment” refers to obtaining desired pharmacological and/or physiological effect. The effect can be therapeutic, which includes achieving, partially or substantially, one or more of the following results: reducing the extent of the disease, disorder or syndrome; ameliorating or improving a clinical symptom or indicator associated with the disorder; and inhibiting or decreasing the likelihood of the progression of the disease, disorder or syndrome.

The term “an effective amount” means an amount of compound (I), or a pharmaceutically acceptable salt thereof, e.g., 0.1 mg to 1000 mg/kg body weight, when administered to a subject, which results in beneficial or desired results, including clinical results, i.e., reversing, alleviating, inhibiting, reducing or slowing the progression of a disease or condition treatable by compound (I), or a pharmaceutically acceptable salt thereof, reducing the likelihood of recurrence of a disease or condition treatable by compound (I), or a pharmaceutically acceptable salt thereof or one or more symptoms thereof, e.g., as determined by clinical symptoms, compared to a control. The expression “an effective amount” also encompasses the amounts which are effective for increasing normal physiological function, for example, between 0.01 mg/kg per day to 500 mg/kg per day.

Another embodiment of the present invention is a pharmaceutical composition comprising at least one compound described herein, or a pharmaceutically acceptable salt thereof, and at least one pharmaceutically acceptable carrier.

Also included are the use of compound (I), or a pharmaceutically acceptable salt thereof in the manufacture of a medicament for the treatment of one or more diseases or conditions described herein. Also included herein are pharmaceutical compositions comprising compound (I), or a pharmaceutically acceptable salt thereof optionally together with a pharmaceutically acceptable carrier, in the manufacture of a medicament for the treatment of one or more diseases or conditions described herein. Also included is compound (I), or a pharmaceutically acceptable salt thereof for use the treatment of a subject with one or more diseases or conditions described herein. Further included are pharmaceutical compositions comprising compound (I), or a pharmaceutically acceptable salt thereof, optionally together with a pharmaceutically acceptable carrier, for use in the treatment of one or more diseases or conditions described herein.

The term “pharmaceutically acceptable carrier” refers to a non-toxic carrier, diluent, adjuvant, vehicle or excipient that does not adversely affect the pharmacological activity of the compound with which it is formulated, and which is also safe for human use. Pharmaceutically acceptable carriers that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5th Ed., a Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003, 20th edition) and in The United States Pharmacopeia: The National Formulary (USP 24 NF19) published in 1999.

A compound (I), or a pharmaceutically acceptable salt thereof, or the compositions of the present teachings may be administered, for example, by oral, parenteral, sublingual, topical, rectal, nasal, buccal, vaginal, transdermal, patch, pump administration or via an implanted reservoir, and the pharmaceutical compositions would be formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

Other forms of administration included in this disclosure are as described in WO 2013/075083, WO 2013/075084, WO 2013/078320, WO 2013/120104, WO 2014/124418, WO 2014/151142, and WO 2015/023915, the contents of which are incorporated herein by reference.

### Pharmaceutical Compositions

The Compound of Formula 1, especially polymorph Tartrate salt Form B is suitable as an active agent in pharmaceutical compositions that are efficacious particularly for the treatment or prevention of diseases, conditions and/or disorders modulated by OCTA inhibition, for example, Alzheimer's disease or related neurodegenerative diseases. The pharmaceutical composition in various embodiments has a pharmaceutically effective amount of the crystalline Compound of Formula 1, especially the polymorph Tartrate salt Form B, along with one or more pharmaceutically acceptable carriers.

As used herein, a “pharmaceutical composition” comprises Tartrate salt Form B and at least one pharmaceutically acceptable carrier, in a unit dose solid form suitable for oral administration (typically a capsule, more particularly a hard gelatin capsule). A list of pharmaceutically acceptable carriers can be found in Remington's Pharmaceutical Sciences.

Thus, in one aspect, provided herein is a pharmaceutical composition comprising polymorph. Tartrate salt Form B of the Compound of Formula 1. In one embodiment, the pharmaceutical composition comprises the polymorph Tartrate salt Form B of the Compound of Formula 1 and at least one pharmaceutically acceptable carrier.

### Definitions

As used herein, the terms “Compound 1”, “Cmpd 1”, “Compound of Formula 1” refer to N-(4-fluoro-5-(((2S,4R)-4-((6-methoxypyrimidin-4-yl)oxy)-2-methylpyrrolidin-1-yl)methyl)thiazol-2-yl)acetamide and having the following structural formula:

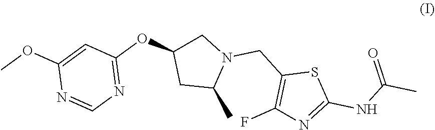 [SMILES: COc1cc(O[C@@H]2C[C@H](C)N(Cc3sc(NC(C)=O)nc3F)C2)ncn1]

In Example using an alternative chemical naming format, “Compound 1” is also referred to as N-(4-fluoro-5-(((2S,4R)-4-((6-methoxypyrimidin-4-yl)oxy)-2-methylpyrrolidin-1-yl)methyl)thiazol-2-yl)acetamide.

As used herein, “crystalline Form A”, “polymorph Form A” and “Form A” are used interchangeably and have no difference in meaning.

As used herein, “crystalline Form B”, “polymorph Form B” and “Form B” are used interchangeably and have no difference in meaning.

As used herein the term “Free Form” or “Freeform” refers to the compound per se without salt.

As used herein, the term “pharmaceutically acceptable carrier” includes any and all solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

As used herein, the term “Alzheimer's disease” or “AD” encompasses both preclinical and clinical Alzheimer's disease unless the context makes clear that either only preclinical Alzheimer's disease or only clinical Alzheimer's disease is intended.

As used herein, the term “treatment of Alzheimer's disease” refers to the administration of the Compound of Formula 1, especially polymorph Form A, to a patient in order to ameliorate at least one of the symptoms of Alzheimer's disease.

As used herein, the term “prevention of Alzheimer's disease” refers to the prophylactic treatment of AD; or delaying the onset or progression of AD.

### List of Abbreviations

ACN acetonitrile
APP amyloid precursor protein
Aβ beta-amyloid peptide
aq. aqueous
Boc₂O Di-cert-butyl dicarbonate
b.p. boiling point
BuLi or nBuLi n-butyllithium
C concentration
CI confidence interval
CDCI₃ deuterated chloroform
cone. concentrated
CSF cerebrospinal fluid
Cu₂O copper(I) oxide
d day,
δ chemical shift in ppm
DCM dichloromethane
DMF A/,A/-dimethylformamide
DMSO dimethylsulfoxide
DSC differential scanning calorimetry
EDC 1-(3-dimethylaminopropyl)-3-ethylcarbodiimide hydrochloride
ESI electrospray ionisation
EtOAc ethyl acetate
g gram
h, hr hour(s)
HCI hydrochloric acid.
Hex. hexane
HOAt 1-hydroxy-7-azabenzotriazole
HPLC, LC high-performance liquid chromatography, liquid chromatography
IPAc isopropyl acetate
K₂CO₃ potassium carbonate
kJ kilojoule
kg kilogram
KOtBu potassium tert-butoxide
kV kilovolt
LC-MS/MS tandem mass spectrometry
mA milliampere
mDSC modulated differential scanning calorimetry
MeOH methanol
MHz megahertz
min minute
ml/mL milliliter
mm millimeter
μI microliter
μηι micrometer
μM micromolar
μmol micromoles
min minute(s)
mmol millimoles
MS mass spectrometry
NaHCO₃ sodium bicarbonate
Na₂SO₄ sodium sulfate
NEt₃ triethylamine
nm nanometer
nM nanomolar
NMR nuclear magnetic resonance spectrometry
PI pharmaceutical intermediate
PK pharmacokinetic
ppm parts per million
q.d. or QD (plaque die
Rf retention factor
RH relative humidity
rpm revolutions per minute
Rt retention time (min)
RT, rt room temperature
s second
SD single dose
Abbreviation Description
T time
TBME tort-butyl methyl ether
TFA trifluoroacetic acid
TGA thermogravimetric analysis
THF tetrahydrofuran
TLC thin layer chromatography
UPLC ultra performance liquid chromatography
v/v by volume
w/w by weight
WL copper Ka radiation wavelength (h_{Cu}=1.5406 A)
wt weight ratio based on the quantity of starting material
XRPD x-ray powder diffraction

### Examples

The following Examples illustrate various aspects of the invention. Examples 1 and 2 show how Compound 1 may be prepared and how it may be crystallized to produce Form A. Example 3 shows how Compound 1 may be prepared and how it may be crystallized to produce Form B. Example 4 describe the XRPD and DSC analysis of HCl Form A. Example 5 describes the phosphate Form A and the corresponding XRPD data. Example 6 describe Tartrate Form B and the corresponding XRPD data.

The preparation of Compound (I) is described in PCT/US2019/051661. (Example 1-22). Compound (I) may also be prepared as described below,

### Example 1

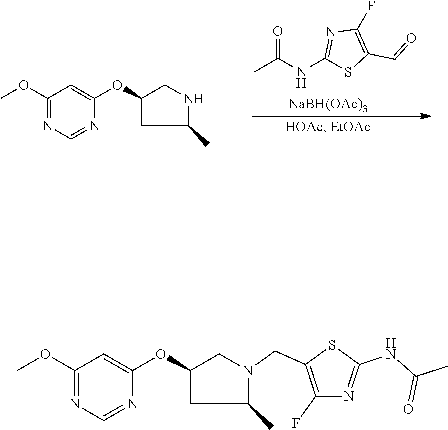

N-(4-Fluoro-5-(((2S,4R)-4-((6-methoxypyrimidin-4-yl)oxy)-2-methylpyrrolidin-1-yl)methyl)thiazol-2-yl)acetamide: To a mixture of the crude 4-methoxy-6-[(3R,5S)-5-methylpyrrolidin-3-yl]oxy-pyrimidine trifluoroacetate (1.65 g, 2.81 mmol; and N-(4-fluoro-5-formyl-thiazol-2-yl)acetamide (429 mg, 2.28 mmol, prepared according to the literature procedure described in WO2018/140299A1) in EtOAc (20 mL) was added N,N-diisopropylethylamine (1.19 mL 6.84 mmol). The mixture was heated to 50° C. for 5 minutes and subsequently cooled to room temperature. To the mixture was added sodium triacetoxyborohydride (1.45 g, 6.84 mmol). The mixture was heated to 50° C. for 1 h, then cooled to room temperature. To the mixture was added saturated NaHCO₃(aq) and EtOAc. The aqueous layer was removed and back-extracted with EtOAc. The combined organics were washed with brine, dried over MgSO₄, filtered, and concentrated in vacuo. The residue was triturated with heptane/EtOAc to provide a pink solid (329 mg). The mother liquor was concentrated in vacuo and the residue was purified over SiO₂ (50% EtOAc/heptane) to provide a yellow solid (98 mg). The solid material (427 mg) was dissolved in MeOH (30 mL) and treated with charcoal. The suspension was filtered over celite and the eluent was concentrated in vacuo to provide the title compound (402 mg, yield 46%). LCMS (ESI): [M+H] 382. ¹H NMR (400 MHz, METHANOL-d₄) δ 8.35 (s, 1H), 6.13 (s, 1H), 5.21-5.47 (m, 1H), 3.85-4.03 (m, 4H), 3.55 (d, J=14.56 Hz, 1H), 3.13 (d, J=11.29 Hz, 1H), 2.47-2.73 (m, 3H), 2.17 (s, 3H), 1.52-1.72 (m, 1H), 1.23 (d, J=5.52 Hz, 3H).
OR
   N-(4-Fluoro-5-(((2S,4R)-4-((6-methoxypyrimidin-4-yl)oxy)-2-methylpyrrolidin-1-yl)methyl)thiazol-2-yl)acetamide: Sodium triacetoxyborohydride (100.3 g, 473.1 mmol) was added to a mixture of 4-methoxy-6-[(3R,5S)-5-methylpyrrolidin-3-yl]oxy-pyrimidine (33 g, 158 mmol) and acetic acid (18.9 g, 315 mmol, 18.0 mL) in EtOAc (743 mL) at 40° C. After 5 min, N-(4-fluoro-5-formyl-thiazol-2-yl)acetamide (30.7 g, 163 mmol) was added to the mixture. After 2 h at 40° C., the mixture was cooled to rt and stirred overnight. A solution of 1N HCl (315 mL) was slowly added to the reaction. The aqueous layer was separated, and the organic layer was extracted with additional IN HCl (150 mL). The combined HCl layers were treated with 50% NaOH to a final pH ˜11 while being cooled with an ice bath. The mixture was extracted with DCM and the organics were dried over MgSO₄, filtered, and concentrated in vacuo. The residue was triturated with MeOH to afford a pink solid. The solid was purified in two batches over SiO₂ (220 g, 20%→60% heptane/(3:1 EtOAc:EtOH 2% NH₄OH) to afford the title compound (29 g, 48% yield). LCMS (ESI): [M+H] 382. ¹HNMR: (500 MHz, CDCl₃) δ 11.16 (br s, 1H), 8.36-8.41 (m, 1H), 6.04-6.08 (m, 1H), 5.28-5.39 (m, 1H), 3.98 (d, J=14.6 Hz, 1H), 3.89-3.94 (m, 3H), 3.64 (d, J=14.6 Hz, 1H), 3.16 (d, J=11.1 Hz, 1H), 2.65 (dd, J=11.1, 6.1 Hz, 1H), 2.48-2.57 (m, 2H), 2.29-2.34 (m, 3H), 1.60-1.72 (m, 2H), 1.20-1.29 (m, 4H). ¹⁹FNMR: (471 MHz, CDCl₃) δ−116 (s, 1F).

### Example 2: Free Form Type A

Free Form Type A is the original form obtained upon synthesis. it also remained unchanged upon exposure to different conditions which indicates that Free form Type A is a stable form.

### Example 3: Free Form Type B

Freeform Type B was obtained via fast cooling method in MeOH. The XRPD pattern was displayed in FIG. 2. TGA/DSC curves displayed in FIG. 2B showed a weight loss of 2.9% up to 150° C. and one endotherm at 162.1° C. (onset temperature). Based on the low TGA weight loss and single DSC endotherm, freeform Type B was postulated to be an anhydrate.

### Example 4: Hydrochloric Acid Form Type A

1. Weigh 700.1 mg freeform into a 50-mL vial, followed by addition of 25 mL acetone to dissolve the freeform; 2. Add 154.0 μL HCl (12 mol/L) into the clear solution slowly with stirring, and precipitation was observed;
3. Stir the mixture at 1000 rpm at RT for 1 day, XRPD result showed HCl salt Type A was obtained;
4. Isolate the solids by filtration and dry the sample under vacuum at RT for 2 days and at 50° C. overnight;
650.6 mg of solid was obtained

### Example 5: Phosphate Form Type A

1. Weigh 700.2 mg freeform into a 50-mL vial, followed by addition of 25 mL acetone to dissolve the freeform;
2. Add 132 μL H3PO4 (15 mol/L) into the clear solution slowly with stirring, and precipitation was observed;
3. Stir the mixture at 1000 rpm at RT for 1 day, XRPD result showed phosphate Type A was obtained;
4. Isolate the solids by filtration and dry the sample under vacuum at RT for 2 days; 819.2 mg of solid was obtained

### Example 6: Tartrate Form Type B

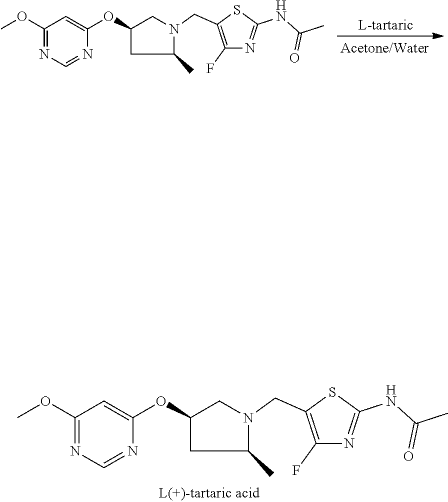 [0258] 1. Weigh 100.0 mg freeform into a 20-mL vial, followed by addition of 4 mL acetone to dissolve the freeform;
2. Weigh 39.6 mg L-tartaric acid into a 3-mL vial, followed by addition of 2 mL acetone to dissolve the acid;
3. Add the L-tartaric acid solution into the freeform solution, and precipitation was observed after stirring for ˜1 hour;
4. Stir the mixture at 1000 rpm at RT for 6 hours, XRPD result showed tartrate Type B was obtained;
5. Isolate the solids by centrifugation (10000 rpm, 2 min) and dry the sample under vacuum at RT for 2 days;
6. 126.9 mg of solid was obtained.

### 1.1 Instruments and Methods

### 1.1.1 XRPD

For XRPD analysis, PANalytical Empyrean/X'Pert3 X-ray powder diffractometers were used. The XRPD parameters used are listed in Table 1-1.

TABLE 1-1
Parameters for XRPD test

| Parameters | Empyrean | X′ Pert3 |
|---|---|---|
| X-Ray wavelength | Cu, Kα; | Cu, Kα; |
| | Kα1 (Å): 1.540598 | Kα1 (Å): 1.540598 |
| | Kα2 (Å): 1.544426 | Kα2 (Å): 1.544426 |
| | intensity ratio | intensity ratio |
| | Kα2/Kα1: 0.50 | Kα2/Kα1: 0.50 |
| X-Ray tube setting | 45 kV, 40 mA | 45 kV, 40 mA |
| Divergence slit | Automatic | 1/8° |
| Scan mode | Continuous | Continuous |
| Scan range (2θ/°) | 3°~40° | 3°~40° |
| Step size (2θ/°) | 0.0167 | 0.0263 |
| Scan step time (s) | 18 | 50 |
| Test time (s) | ~5 min 30 s | ~5 min |

### 1.1.2 TGA/DSC

TGA data were collected using a TA Q500/Q5000 TGA from TA Instruments. DSC was performed using a TA Q200/Q2000 DSC from TA Instruments. Detailed parameters used are listed in Table 1-2.

TABLE 1-2
Parameters for TGA and DSC test

| Parameters | TGA | DSC |
|---|---|---|
| Method | Ramp | Ramp |
| Sample pan | Aluminum, open | Aluminum, crimped |
| Temperature | RT- desired temperature | 25° C. - desired temperature |
| Heating rate | 10° C./min | 10° C./min |
| Purge gas | N₂ | N₂ |

### Salt Screening

A total of 108 polymorph screening experiments were performed for compound (I) freeform. Based on X-ray powder diffraction (XRPD) comparison, two crystal forms (freeform Type A and Type B) were discovered and further characterized by thermogravimetric analysis (TGA) and differential scanning calorimetry (DSC), which suggested that both forms were anhydrates. Competitive slurry experiments indicated that Type A was thermodynamically more stable than Type B from RT to 50° C.
According to the approximate solubility of freeform starting material and predicted pKa values, salt screening was performed under 315 conditions using 31 acids/bases (2 charging ratios for 4 acids) in 9 solvent systems. The starting material and corresponding salt former at 1:1, 1:2 or 2:1 molar ratio were added in an HPLC glass vial, followed by addition of 0.5 or 1.0 mL of solvent. The mixtures were then stirred at 1000 rpm at RT for ˜70 hrs, and the resulting suspensions were centrifuged (10000 rpm, 2 mins) to retrieve the solids for vacuum drying at RT. If clear solutions were obtained, the samples were transferred to 5° C. to slurry overnight and the resulting solid was isolated and dried under vacuum at RT overnight. If clear solutions were still obtained, the samples were transferred to evaporate at RT. All the solids were then analyzed by XRPD.

### Salt Re-Preparation

Based on the characterization results (low TGA weight loss, sharp DSC endotherm at high temperature) and safety class of salt former, HCl salt Type A, phosphate Type A and tartrate Type B were selected for re-preparation, which were successfully obtained via solution crystallization at 50/100-mg scale and further 700-mg scale. The re-prepared salts at 700-mg scale were characterized by XRPD, TGA, DSC, and HPLC/IC, and the characterization results were summarized in Table and 1-4.

TABLE 1-3
Characterization of re-prepared salts at 700-mg scale

| | | Weight | Endotherm | Stoichiometric | Residual |
|---|---|---|---|---|---|
| | | loss (%, | (° C., | ratio | solvent |
| Ex. | Form | 150° C.) | onset) | (FF/acid) | (wt %) |
| | HCl salt | 0.4 | 198.5 | 1:1.0 | 1.8 |
| | Type A | | | | (Acetone) |
| | Phosphate | 0.8 | 181.5 | 1:1.1 | 0.2 |
| | Type A | | | | (Acetone) |
| | Tartrate | 0.8 | 186.8 | 1:1.1 | 0.8 |
| | Type B | | | | (Acetone) |

TABLE 1-4
Summary of solid stability data for freeform Type A

| | | | | | Purity/ |
|---|---|---|---|---|---|
| Starting | | | Form | Purity | Initial |
| material | Condition | Time point | change | (Area %) | (%) |
| Freeform | NA | Initial | NA | 98.92 | NA |
| Type A | 25° C./ | 2 weeks | No | 98.94 | 100.0 |
| | 60% RH | 1 month | No | 98.99 | 100.1 |
| | 40° C./ | 1 week | No | 98.91 | 100.0 |
| | 75% RH | 2 weeks | No | 98.99 | 100.1 |
| | | 1 month | No | 98.98 | 100.0 |
| | White Light | Dark control | No | 98.98 | 100.0 |
| | | White light* | No | 98.84 | 99.9 |
| | | (ICH) | | | |
| | UV light | Dark control | No | 99.01 | 100.1 |
| | | UV light** | No | 98.85 | 99.9 |
| | | (ICH) | | | |
| HCl salt | NA | Initial | NA | 99.07 | NA |
| Type A | 25° C./ | 1 month | No | 99.02 | 99.9 |
| | 60% RH | | | | |
| | 40° C./ | 1 month | No | 99.02 | 99.9 |
| | 75% RH | | | | |
| | White Light | Dark control | No | 99.18 | 100.1 |
| | | White light* | No | 99.13 | 100.1 |
| | | (ICH) | | | |
| | UV light | Dark control | No | 99.13 | 100.1 |
| | | UV light** | No | 99.19 | 100.1 |
| | | (ICH) | | | |
| Phosphate | NA | Initial | NA | 99.12 | NA |
| Type A | 25° C./ | 1 month | No | 99.28 | 100.2 |
| | 60% RH | | | | |
| | 40° C./ | 1 month | No | 99.30 | 100.2 |
| | 75% RH | | | | |
| | White Light | Dark control | No | 99.25 | 100.1 |
| | | White light* | No | 99.22 | 100.1 |
| | | (ICH) | | | |
| | UV light | Dark control | No | 99.25 | 100.1 |
| | | UV light** | No | 99.21 | 100.1 |
| | | (ICH) | | | |
| Tartrate | NA | Initial | NA | 99.14 | NA |
| Type B | 25° C./ | 1 month | No | 99.08 | 99.9 |
| | 60% RH | | | | |
| | 40° C./ | 1 month | No | 99.18 | 100.0 |
| | 75% RH | | | | |
| | White Light | Dark control | No | 99.23 | 100.1 |
| | | White light | No | 99.22 | 100.1 |
| | | (ICH) | | | |
| | UV light | Dark control | No | 99.22 | 100.1 |
| | | UV light* | No | 99.22 | 100.1 |
| | | (ICH) | | | |

*White light: 1.2 million lux hour
**UV light: 200 W · hrs/m²

1. A crystalline form of formula (I), wherein the compound is N-(4-fluoro-5-(((2S,4R)-4-((6-methoxypyrimidin-4-yl)oxy)-2-methylpyrrolidin-1-yl)methyl)thiazol-2-yl)acetamide

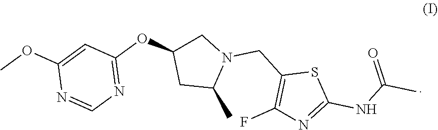 [SMILES: COc1cc(O[C@@H]2C[C@H](C)N(Cc3sc(NC(C)=O)nc3F)C2)ncn1]

2. The crystalline form according to claim 1 comprising Form A.

3. The crystalline form according to claim 1-2 consisting essentially of Form A.

4. The crystalline form according to claim 3, wherein said Form A is in substantially pure form.

5. The crystalline form according to claim 1 comprising Form B.

6. The crystalline form according to claim 1 or 5 consisting essentially of Form B.

7. The crystalline form according to claim 6, wherein said Form B is in substantially pure form.

8. The crystalline form according to claims 1-4, wherein said Form A is anhydrate Freeform.

9. The crystalline form according to claims 1-4, wherein said Form A is anhydrate hydrochloric salt Form.

10. The crystalline form according to claims 1-4, wherein said Form A is anhydrate Phosphate salt Form.

11. The crystalline form according to claims 1, 5-7, wherein said Form B is anhydrate Tartrate salt Form.

12. The crystalline form according to claim 2 or 8 characterized by a x-ray diffraction powder diffraction pattern comprising four or more 2θ values selected from the group consisting of wherein anhydrate Free Form A of the compound according to claims 2 or 8 which has an X-ray powder diffraction pattern with at least one two or three peaks having an angle of refraction 2 theta (θ) values selected from 4.3, 8.6 and 12.0° when measured using CuKa radiation, wherein said values are plus of minus 0.2° 2θ.

13. The crystalline anhydrate Free Form A of the compound according to claim 2 or 8 which has an X-ray powder diffraction pattern with at least four peaks having an angle of refraction 2 theta (θ) values selected from 13.5, 14.9, 21.1, 24.4 and 27.2° when measured using CuKa radiation, wherein said values are plus of minus 0.2° 2θ.

14. The crystalline Free Form A of the compound according to claim 2 or 8 which has an X-ray powder diffraction pattern with at least five peaks having an angle of refraction 2 theta (θ) values selected from 4.3, 8.6, 10, 11, 12, 13.5, 14.9, 19.9, 21.1, 24.4° when measured using CuKa radiation, wherein said values are plus of minus 0.2° 2θ.

15. The crystalline anhydrate Free Form A of the compound according to claims 2 or 8 which has an X-ray powder diffraction spectrum substantially the same as the X-ray powder diffraction spectrum shown in FIG. 1.

16. The crystalline form according to claim 2 or 9 characterized by a x-ray diffraction powder diffraction pattern comprising four or more 2θ values selected from the group consisting of wherein anhydrate Hydrochloric acid Form A of the compound according to claims 2 or 9 which has an X-ray powder diffraction pattern with at least one two or three peaks having an angle of refraction 2 theta (θ) values selected from 9.6, 15.6, 21.5, 23.6 when measured using CuKa radiation, wherein said values are plus of minus 0.2° 2θ.

17. The crystalline anhydrate Hydrochloric acid Form A of the compound according to claim 2 or 9 which has an X-ray powder diffraction pattern with at least four peaks having an angle of refraction 2 theta (θ) values selected from 9.6, 15.6, 17.1, 20.4, 21.5, 23.6, 26.5 when measured using CuKa radiation, wherein said values are plus of minus 0.2° 2θ.

18. The crystalline anhydrate Hydrochloric acid Form A of the compound according to claim 2 or 9 which has an X-ray powder diffraction pattern with at least five peaks having an angle of refraction 2 theta (θ) values selected from 9.6, 10.2, 12.2, 15.2, 15.6, 17.1, 20.4, 21.5, 23.6, 26.5 when measured using CuKa radiation, wherein said values are plus of minus 0.2° 2θ.

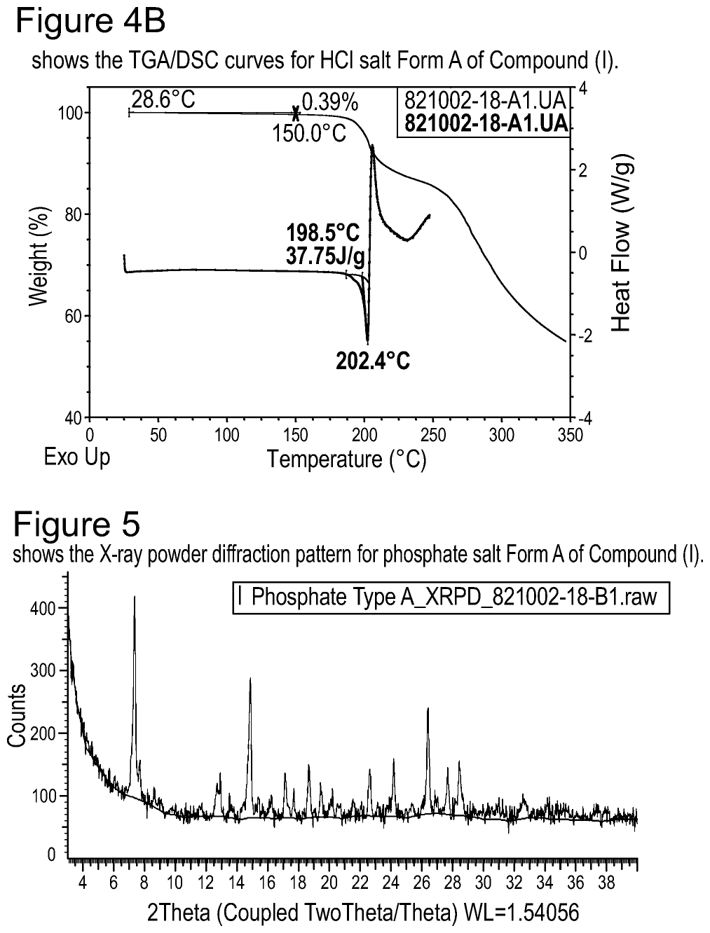

19. The crystalline anhydrate hydrochloric acid Form A of the compound according to claims 2 or 9 which has an X-ray powder diffraction spectrum substantially the same as the X-ray powder diffraction spectrum shown in FIG. 4.

20. The crystalline form according to claim 2 or 10 characterized by a x-ray diffraction powder diffraction pattern comprising four or more 2θ values selected from the group consisting of wherein anhydrate Phosphate Form A of the compound according to claims 2 or 10 which has an X-ray powder diffraction pattern with at least one two or three peaks having an angle of refraction 2 theta (θ) values selected from 7.3, 14.8, 22.5, 24.1, 26.3 when measured using CuKa radiation, wherein said values are plus of minus 0.2° 2θ.

21. The crystalline anhydrate Phosphate Form A of the compound according to claim 2 or 10 which has an X-ray powder diffraction pattern with at least four peaks having an angle of refraction 2 theta (θ) values selected from 7.3, 14.8, 17.1, 18.6, 22.5, 24.1, 26.3, 27.6 when measured using CuKa radiation, wherein said values are plus of minus 0.2° 2θ.

22. The crystalline anhydrate Phosphate Form A of the compound according to claim 2 or 10 which has an X-ray powder diffraction pattern with at least four peaks having an angle of refraction 2 theta (θ) values selected from 7.3, 14.8, 17.1, 17.6, 18.6, 22.5, 24.1, 26.3, 27.6, 28.4 when measured using CuKa radiation, wherein said values are plus of minus 0.2° 2θ.

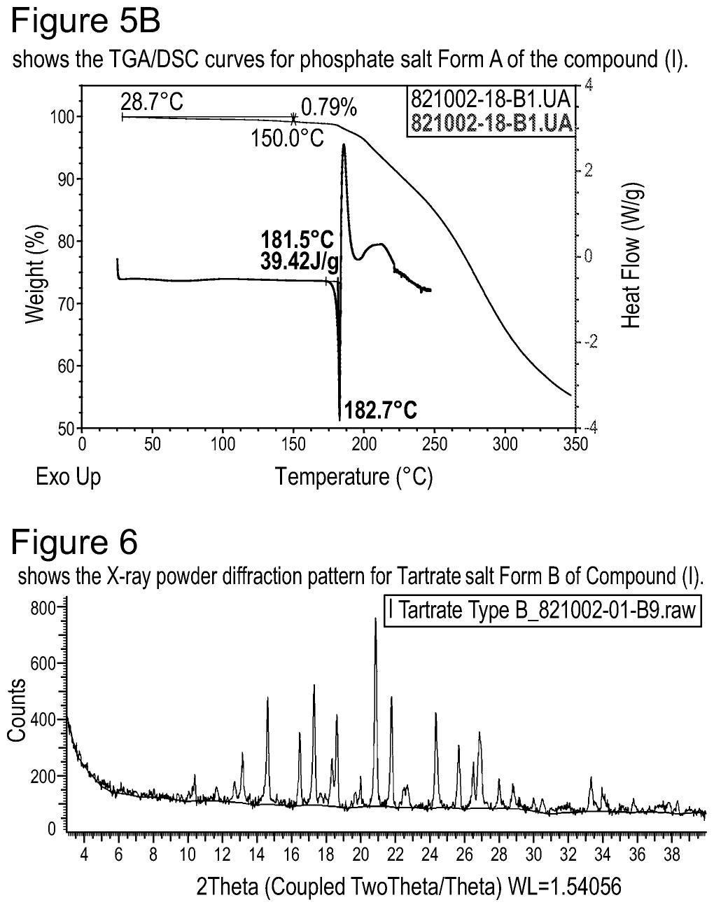

23. The crystalline anhydrate Phosphate Form A of the compound according to claims 2 or 10 which has an X-ray powder diffraction spectrum substantially the same as the X-ray powder diffraction spectrum shown in FIG. 5.

24. The crystalline form according to claim 2 or 11 characterized by a x-ray diffraction powder diffraction pattern comprising four or more 2θ values selected from the group consisting of wherein anhydrate Tartrate Form B of the compound according to claims 2 or 11 which has an X-ray powder diffraction pattern with at least one two or three peaks having an angle of refraction 2 theta (θ) values selected from 12.7, 13.2, 14.6, 17.3, 20.9, when measured using CuKa radiation, wherein said values are plus of minus 0.2° 2θ.

25. The crystalline anhydrate Tartrate Form B of the compound according to claim 2 or 11 which has an X-ray powder diffraction pattern with at least four peaks having an angle of refraction 2 theta (θ) values selected from 12.7, 13.2, 14.6, 17.3, 20.9, 21.8, 24.4 when measured using CuKa radiation, wherein said values are plus of minus 0.2° 2θ.

26. The crystalline anhydrate Tartrate Form B of the compound according to claim 2 or 11 which has an X-ray powder diffraction pattern with at least fivepeaks having an angle of refraction 2 theta (e) values selected from 12.7, 13.2, 14.6, 16.5, 17.3, 20.9, 21.8, 24.4, 25.7, 26.9, 28.8 when measured using CuKa radiation, wherein said values are plus of minus 0.2° 2θ.

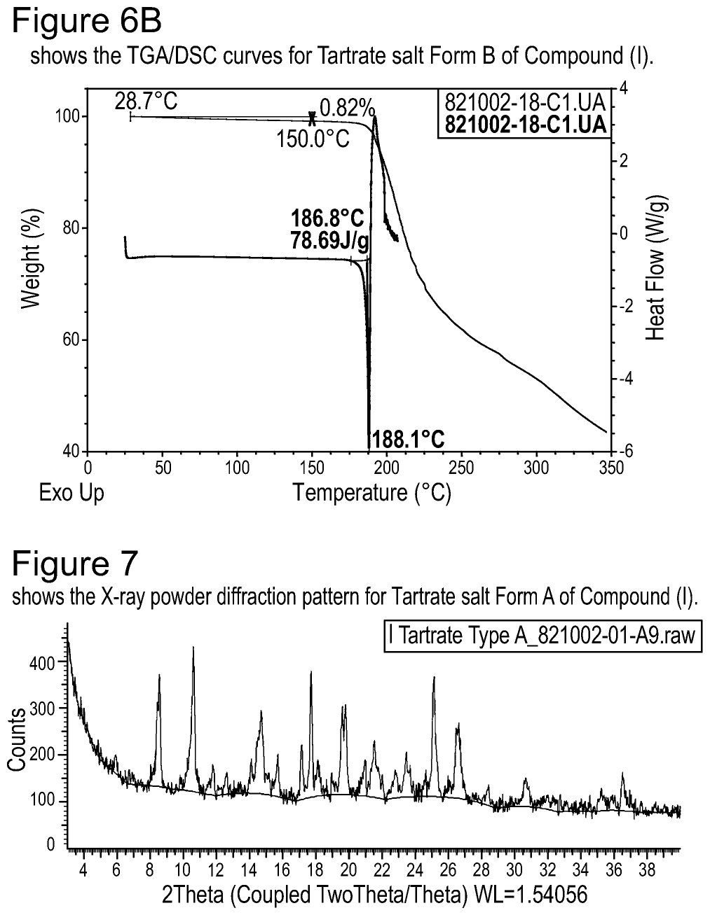

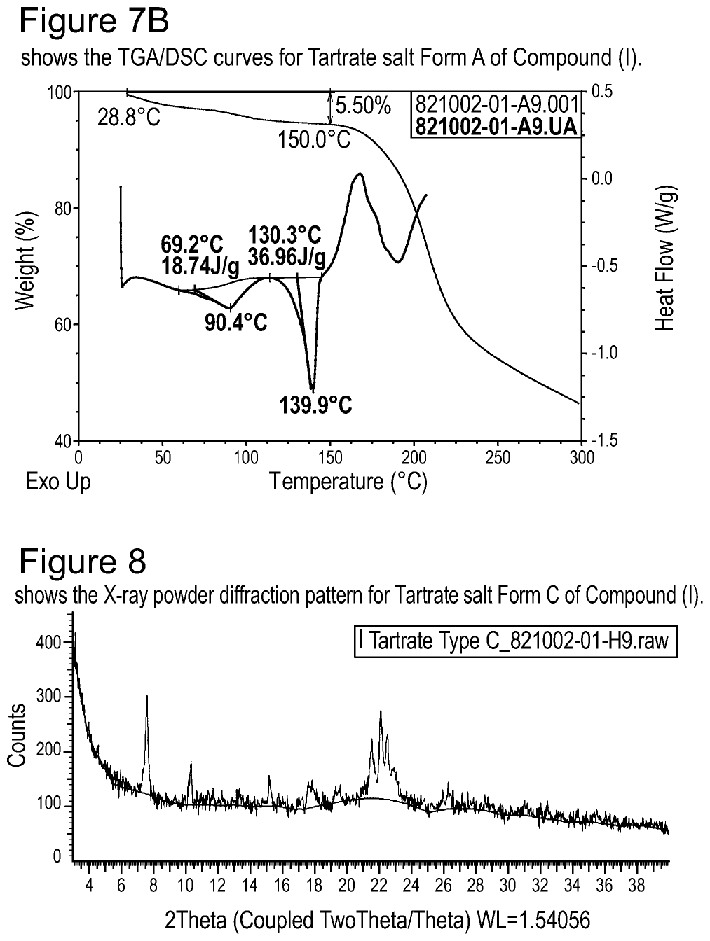

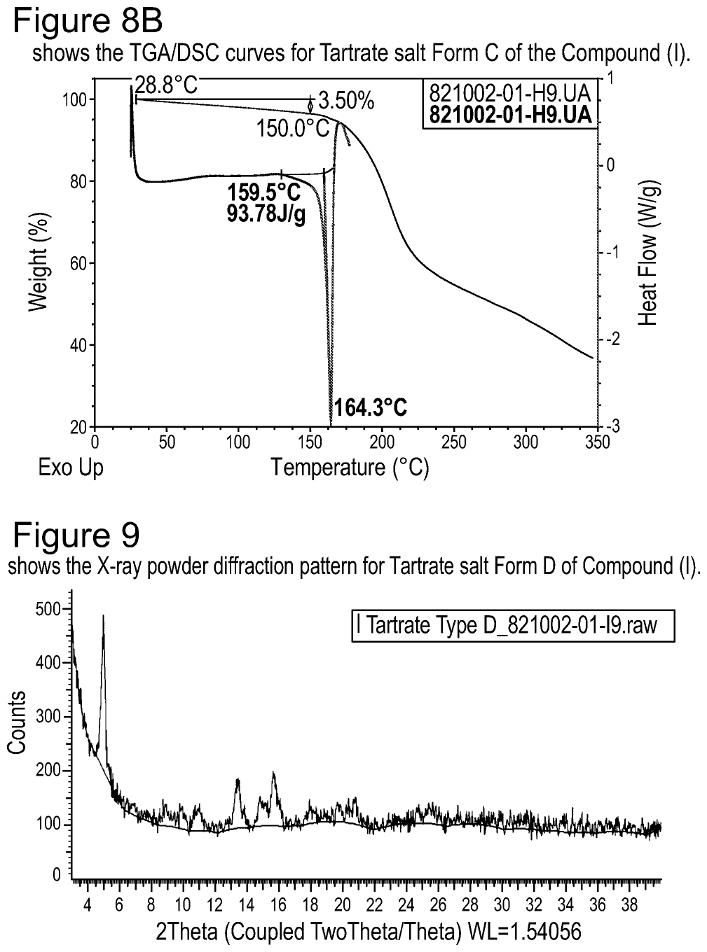

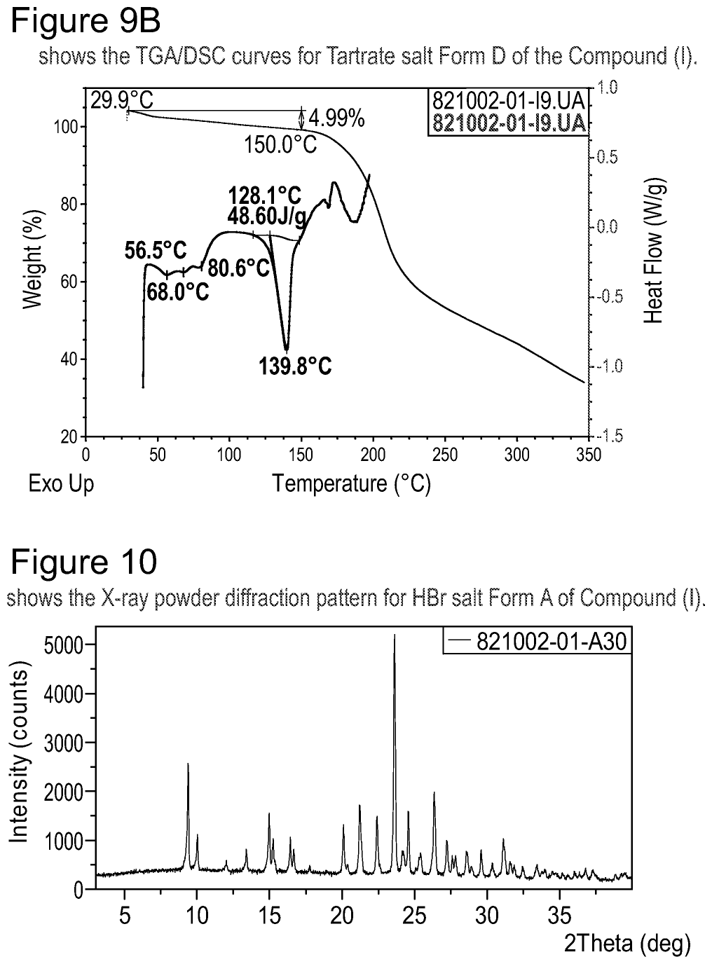

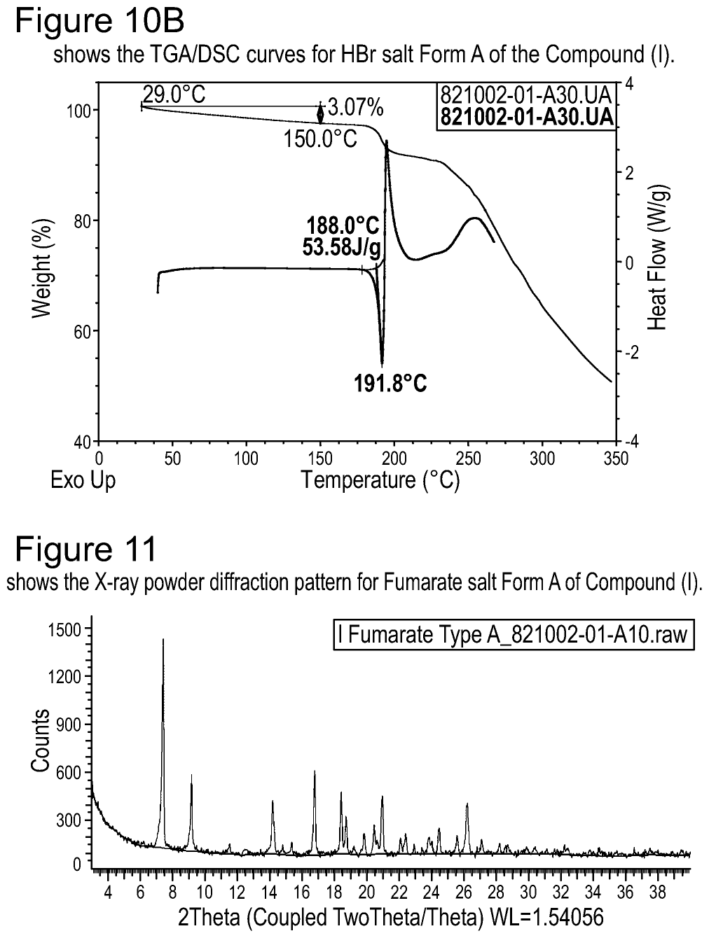

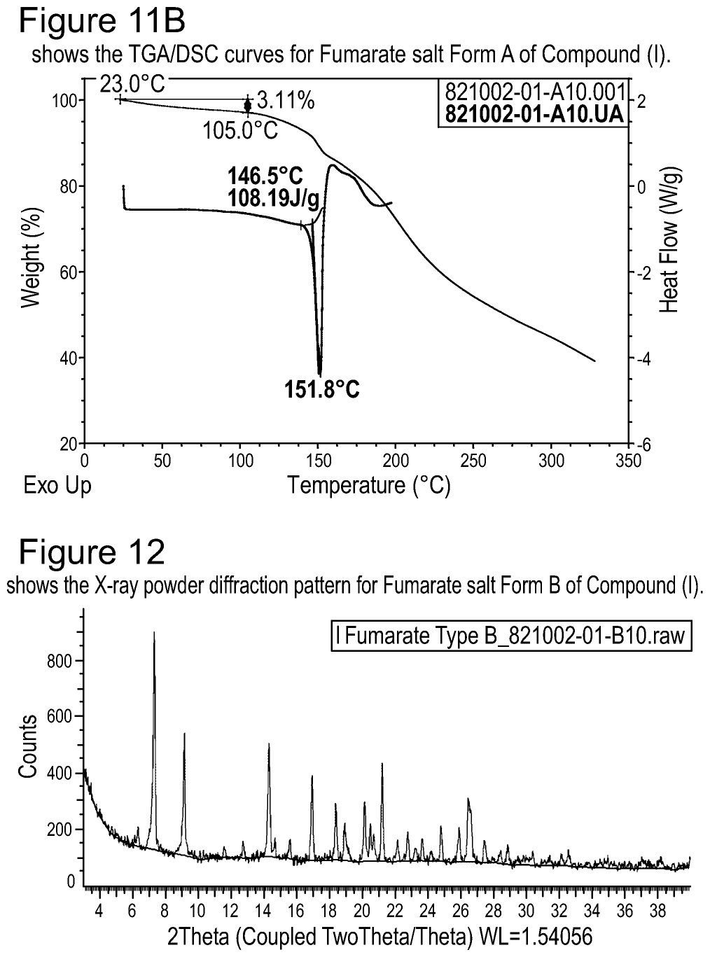

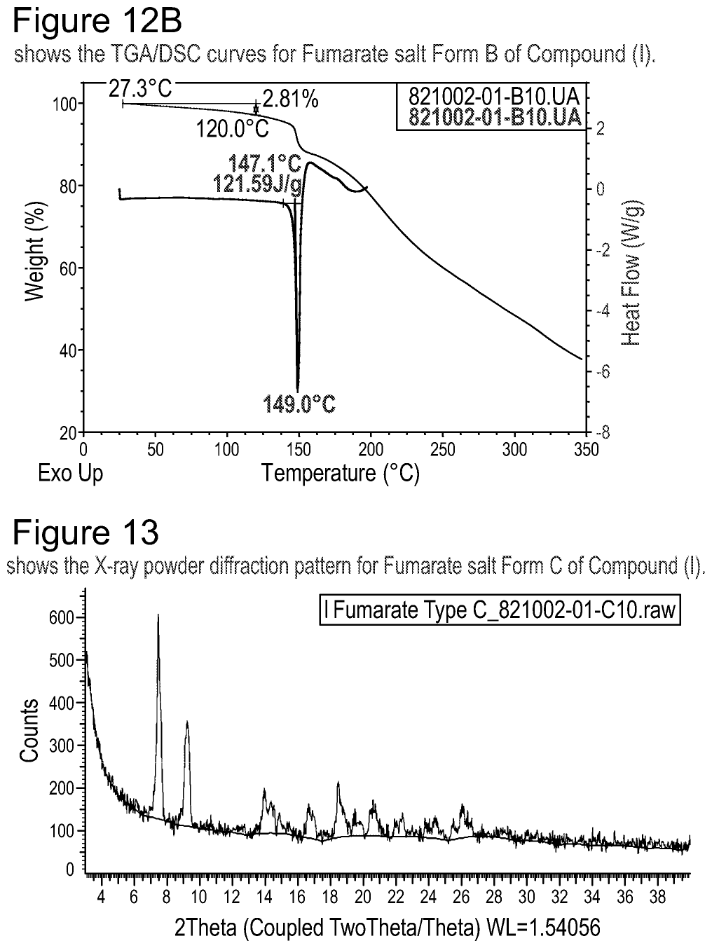

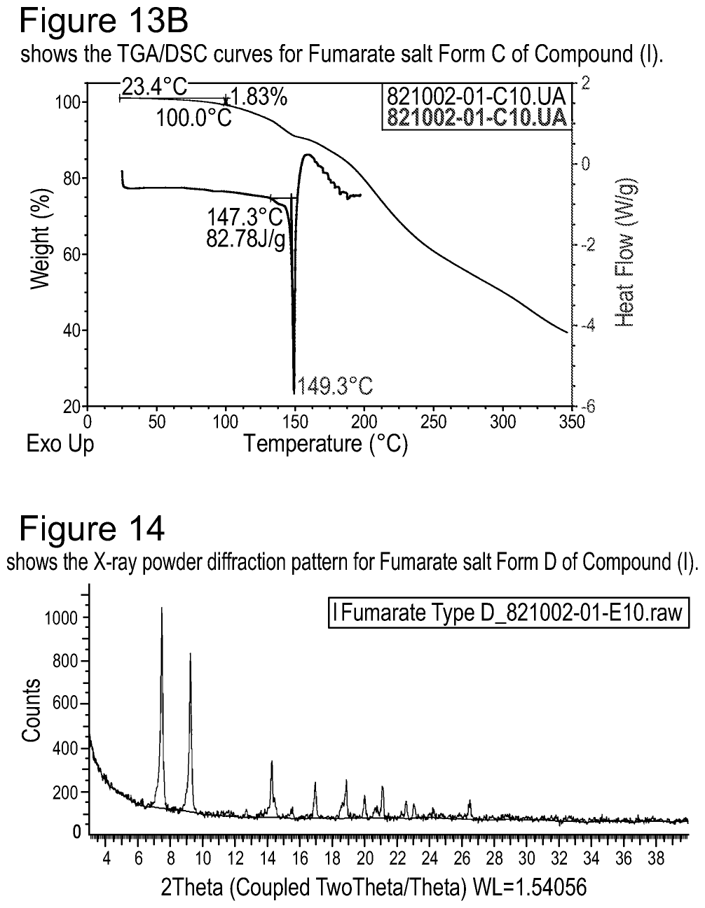

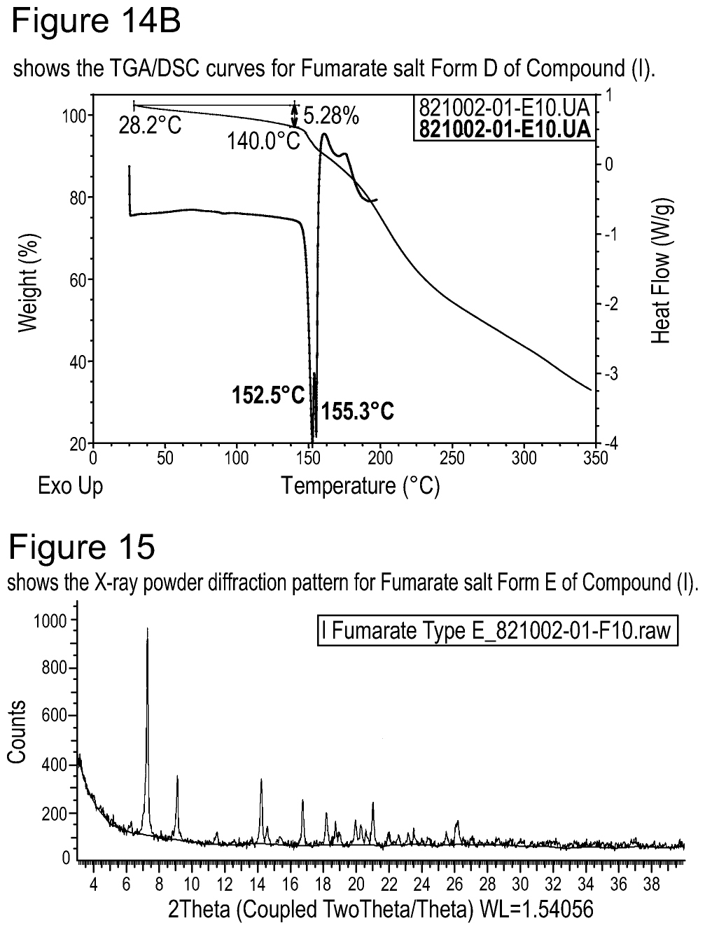

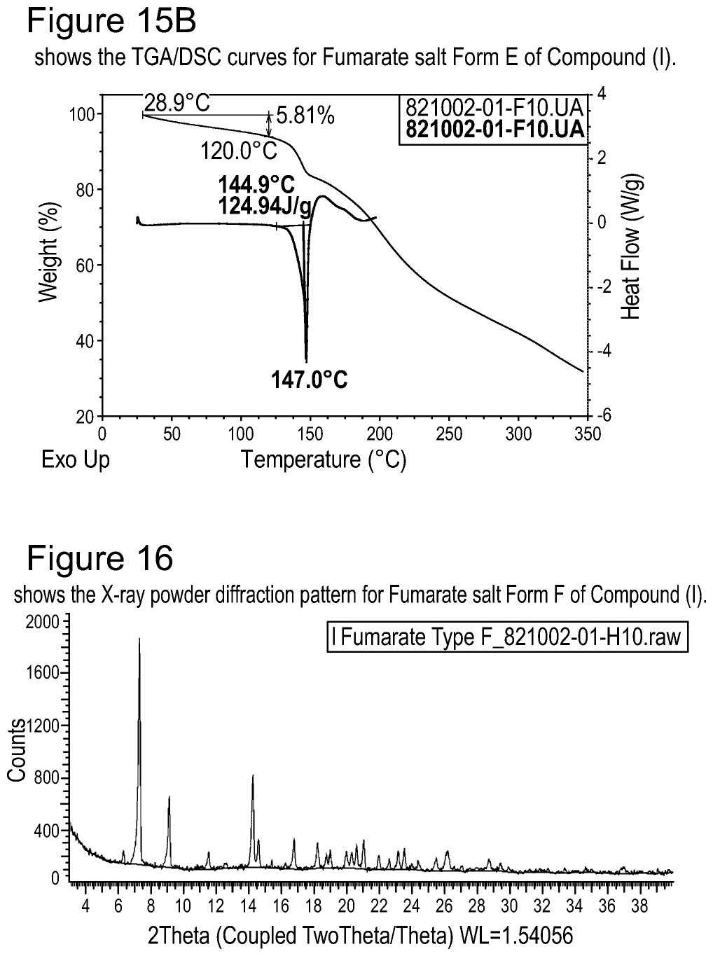

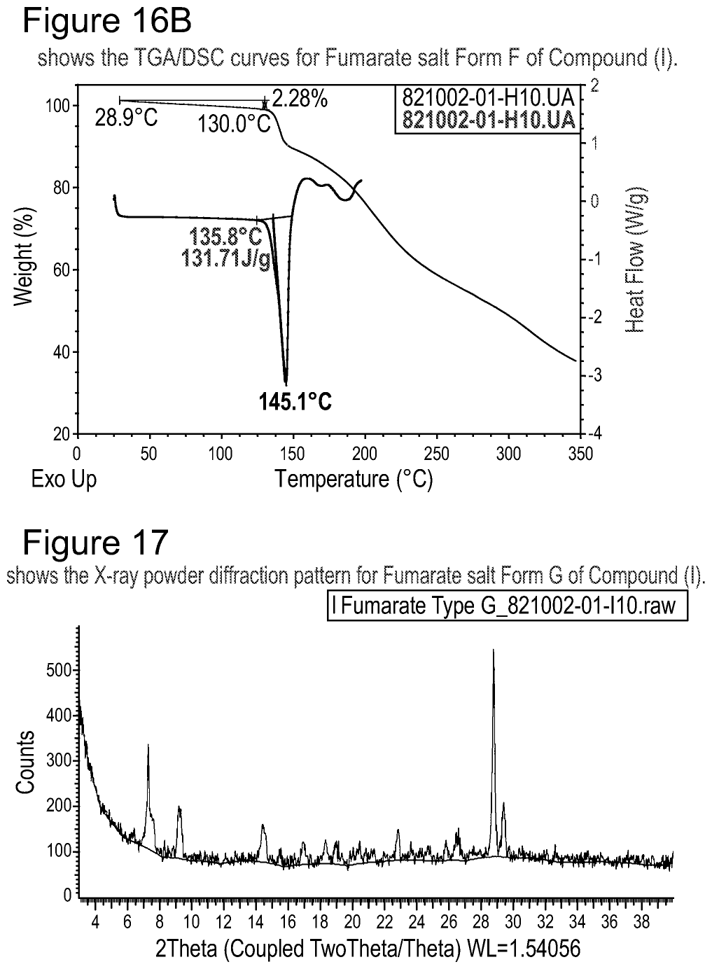

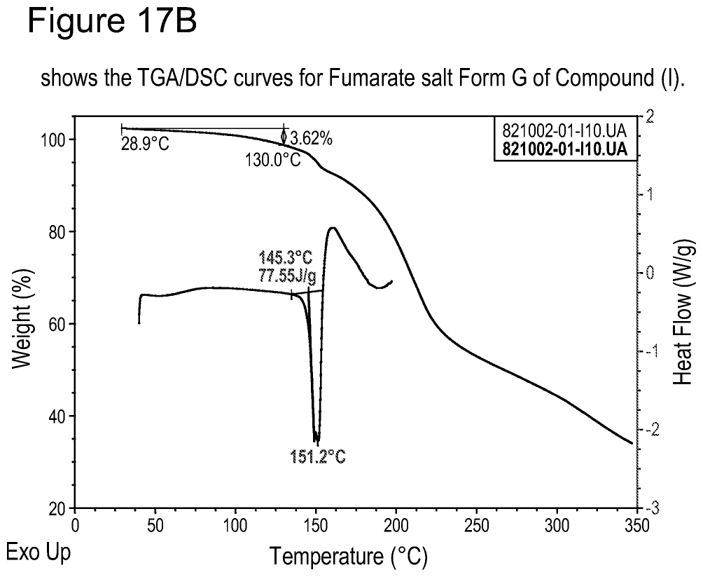

27. The crystalline anhydrate Tartrate Form B of the compound according to claims 2 or 11 which has an X-ray powder diffraction spectrum substantially the same as the X-ray powder diffraction spectrum shown in FIG. 6.

28. The crystalline anhydrate Free Form A of the compound of claim 1, 2 or 8 having a differential scanning calorimetry (DSC) thermogram substantially the same as that shown in FIG. 1B.

29. The crystalline anhydrate hydrochloric acid Form A of the compound of claim 1, 2 or 9 having a differential scanning calorimetry (DSC) thermogram substantially the same as that shown in FIG. 4B.

30. The crystalline anhydrate Phosphate Form A of the compound of claim 1, 2 or 10 having a differential scanning calorimetry (DSC) thermogram substantially the same as that shown in FIG. 5B.

31. The crystalline anhydrate Tartrate Form B of the compound of claim 1, 2 or 11 having a differential scanning calorimetry (DSC) thermogram substantially the same as that shown in FIG. 6B.

32. A pharmaceutical composition comprising the crystalline form according to claims 2 or 8-11 and a pharmaceutically acceptable carrier or diluent.

33. A pharmaceutical composition according to claim 32 wherein the crystalline form is anhydrate Free Form A.

34. The pharmaceutical composition according to claim 33 wherein anhydrate Free Form A is in substantially pure form.

35. The pharmaceutical composition according to claim 32 wherein the crystalline form is anhydrate Hydrochloric acid Form A is in substantially pure form.

36. The pharmaceutical composition according to claim 32 wherein the crystalline form is anhydrate Phosphate Form A is in substantially pure form.

37. The pharmaceutical composition according to claim 32 wherein the crystalline form is anhydrate Tartrate Form B is in substantially pure form.

38. A method of treating Alzheimer's disease in a patient, comprising administering to a patient in need of such treatment an effective amount of a crystalline form of N-(4-fluoro-5-((2S,4R)-4-((6-methoxypyrimidin-4-yl)oxy)-2-methylpyrrolidin-1-yl)methyl)thiazol-2-yl)acetamide according to claim 2.

39. A method of preventing the progression of mild cognitive impairment to Alzheimer's disease in a patient, comprising administering to a patient in need of such treatment an effective amount of a crystalline form of N-(4-fluoro-5-(((2S,4R)-4-((6-methoxypyrimidin-4-yl)oxy)-2-methylpyrrolidin-1-yl)methyl)thiazol-2-yl)acetamide according to claim 2.

40. A method of treating progressive supranuclear palsy in a patient, comprising administering to a patient in need of such treatment an effective amount of a crystalline form of N-(4-fluoro-5-(((2S,4R)-4-((6-methoxypyrimidin-4-yl)oxy)-2-methylpyrrolidin-1-yl)methyl)thiazol-2-yl)acetamide according to claim 2.

41. A method according to claims 38-40 wherein said crystalline form is anhydrate Free Form A.

42. A method according to claims 38-40 wherein said crystalline form is anhydrate hydrochloric acid Form A.

43. A method according to claims 38-40 wherein said crystalline form is anhydrate Phosphate Form A.

44. A method according to claims 38-40 wherein said crystalline form is anhydrate Tartrate Form B.

45. A composition comprising at least 90 weight % of crystalline form according to claim 2, based upon the weight of the composition.

46. The composition of claim 45, wherein the crystalline form is anhydrate Free Form A.

47. The composition of claim 45, wherein the crystalline form is anhydrate Free Form B.
